(19) United States
(12) Patent Application Publication
(10) Pub. No.: US 2023/0248859 A1
(43) Pub. Date: Aug. 10, 2023

(54) PLANT DISINFECTION APPARATUS

(71) Applicant: LESLIE DAVID HOWE, Kanata (CA)

(72) Inventor: LESLIE DAVID HOWE, Kanata (CA)

(73) Assignee: SOUTHPAC TRUST INTERNATIONAL, TRUSTEE OF THE LDH TRUST, Raratonga (CK)

(21) Appl. No.: 17/832,577

(22) Filed: Jun. 4, 2022

Related U.S. Application Data
(60) Provisional application No. 63/307,713, filed on Feb. 8, 2022.
(60) Provisional application No. 63/316,565, filed on Mar. 4, 2022.
(60) Provisional application No. 63/326,540, filed on Apr. 1, 2022.

(51) Int. Cl. A61L 2/10; A61L 2/24

(52) U.S. Cl. CPC A61L 2/10; A61L 2/24; A61L 2202/11; A61L 2202/14

(57) ABSTRACT

In an example embodiment of the disclosed technology, a plant disinfection apparatus is provided that may comprise a support device configured to directly or indirectly support one or more UVC probes beneath the support device. The one or more UVC probes may each comprise a relatively elongated shape comprising a major axis Y, a light source that emits UVC light in a predominantly perpendicular distribution pattern relative to axis Y, and a suspension device configured to directly or indirectly attach to the support device. The UVC probe may be configured to be raised and lowered in a relatively vertical orientation and may further comprise a means of conveying power to the UVC light source. The one or more UVC probes may be configured to be lowered into the foliage of one or more plants disposed beneath the plant disinfection apparatus.

### CROSS REFERENCE TO RELATED APPLICATIONS

This application claims the benefit of the following U.S. Provisional Patent Application, the contents of which are incorporated by reference in their entirety as if set forth in full: U.S. Provisional Patent No. 63/307,713 entitled “Plant Disinfection Apparatus” filed Feb. 8, 2022, U.S. Provisional Patent No. 63/316,565 entitled “Plant Disinfection Apparatus” filed Mar. 4, 2022, and U.S. Provisional Patent No. 63/326,540 entitled “Plant Disinfection Apparatus” filed Apr. 1, 2022.

### TECHNICAL FIELD

This disclosure generally relates to pathogenic reduction systems or methods for plants.

### BACKGROUND

There is a continuing need for systems and methods that can decrease pathogen proliferation in horticultural applications.

### BRIEF DESCRIPTION OF THE FIGURES

FIG. 1 shows a perspective view of an example embodiment of plant disinfection apparatus.

FIG. 2A shows an example embodiment of plant disinfection apparatus wherein a UVC beam is lowered into a position wherein UVC probes are in proximity to the lower portion of plants.

FIG. 2B shows an example embodiment of plant disinfection apparatus wherein a UVC beam is lowered into a position wherein UVC probes are in proximity to the lower portion of plants.

FIG. 3A shows an upper perspective of an example embodiment of plant disinfection apparatus wherein a UVC beam is lowered into a position wherein UVC probes are in proximity to the upper region of plants.

FIG. 3B shows a profile view in the X plane of an example embodiment of plant disinfection apparatus suspended above a plurality of rows of plants which have been defoliated for illustrative purposes.

FIG. 3C shows a profile view in the Y plane of the example embodiment of plant disinfection apparatus shown if FIG. 3B.

FIG. 3D shows a perspective view of the example embodiment of plant disinfection apparatus shown if FIG. 3B.

FIG. 3E shows an example embodiment of a UVC probe comprising a UVC light source, wherein example UVC light rays are emitted on an adjacent plant.

FIG. 4 shows a detailed perspective view of an example embodiment of UVC beam with UVC probes and various elements of a lifting device.

FIG. 5A shows a bottom perspective view of an example embodiment of a control unit.

FIG. 5B shows a top perspective view of an example embodiment of a control unit.

FIG. 6 shows an inside view facing up in an example embodiment of a control unit.

FIG. 7 shows an inside view facing down in an example embodiment of a control unit.

FIG. 8 shows a perspective view of an example embodiment of track system with control unit.

FIG. 9 shows an example embodiment of control unit mounted to a track system.

FIG. 10 shows a partial cutaway profile view of the same example embodiment shown in FIG. 9

FIG. 11 shows a closer detailed view of an example embodiment of power feed system.

FIG. 12 shows a perspective view of an example embodiment of plant disinfection apparatus with a novel power feed system and is shown without the UVC beam.

FIG. 13 shows a close-up perspective view of the control unit and power feed system of the example embodiment of plant disinfection apparatus shown in FIG. 12

FIG. 14 shows a different close-up perspective view of the control unit and power feed system of the example embodiment of plant disinfection apparatus shown in FIG. 12

FIG. 15 shows a close-up perspective view of the control unit with pivoting cable clamp of the example embodiment of plant disinfection apparatus shown in FIG. 12

FIG. 16A shows a profile view of an example embodiment of UVC probe attached to a UVC beam, wherein the UVC probe comprises a cutaway view of a penetration device and a transition device.

FIG. 16B shows a profile view of an example embodiment of UVC probe attached to a UVC beam as shown in FIG. 16A, wherein the UVC probe comprises a standard view of a penetration device and a transition device.

FIG. 17 shows a perspective view if FIG. 16B.

FIG. 18 shows an example embodiment of plant disinfection apparatus.

FIG. 19 shows a perspective view of an example embodiment of power feed system for moving apparatuses.

FIG. 20 shows a perspective of an example embodiment of an anti-sway apparatus.

FIG. 21A shows a profile view of a UVC light source.

FIG. 21B shows the UVC light source shown in FIG. 20A comprising an example embodiment of UVC probe wherein a cutaway view of a penetration device attached to the lower tip of the UVC light source, a cutaway view of a transition device attached to the top of the UVC light source, and a hollow tube suspension device attached to the transition device are all shown.

FIG. 21C shows the UVC light source shown in FIG. 20A comprising an example embodiment of UVC probe wherein a standard view of a penetration device attached to the lower tip of the UVC light source, a standard view of a transition device attached to the top of the UVC light source, and a hollow tube suspension device attached to the transition device are all shown.

FIG. 22A shows a profile view of a commercially available submersible UVC light source with a curved bottom tip.

FIG. 22B shows a profile view of a commercially available submersible UVC light source with a bottom end cap.

### DETAILED DESCRIPTION

Although various embodiments of the invention may be described with respect to cultivating cannabis, this may be for illustrative purposes only, and should not be construed to limit the scope of possible applications for the various embodiments of the invention. For example, implementations of the disclosed technologies may apply to other crops such as tomatoes etc.

The written descriptions may use examples to disclose certain implementations of the disclosed technology, including the best mode, and may also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Plant pathogens may be a major issue for cannabis growers. Pathogens may reduce or eliminate crop yields which can be costly for growers. Additionally, it can be costly for growers to apply various mitigation techniques to control pathogens. One of the mitigation techniques may be the use of various pesticides. However, health concerns and governmental regulations may limit or curtail the use of many different pesticides. Some major cannabis pathogens may include White Powdery Mildew (Fungi Golovinomyces), Gray Mold aka. Bud Rot (Fungi Botrytis), Fusarium Wilt & Root Rot (Fungi Fusarium), Spider mites (Arachnid Tetranychus urticae), Damping Off & Pythium Rot (Fungi/Protist Pythium).

The negative impact from the use of chemicals, in addition to extra costs, can include plant stress, pathogen resistance to chemical treatments and interference with biocontrol of diseases that may be kept in check by naturally occurring microflora. More importantly, they may not be eco-friendly. There may be a movement within the cannabis growing industry to develop more sustainable and eco-friendly agricultural practices, with the intention of becoming chemical-free.

Pathogens may occur not only in the plants and growing media, but also may be present and spread from people, as well as any surface in the vicinity of the plants. Great care may usually be taken in cannabis growing facilities to regularly clean and disinfect surfaces, and for people to take precautionary measures to lower the risk of spreading pathogens from outside the growing area. Prevention, as well as elimination of these pathogens can be effectively accomplished with UVC light (perhaps anywhere in the range of 222 nm to 290 nm) given that a sufficient UVC dosage is administered. UVC may have been used for pathogen disinfection for over 100 years. The science is well established and will therefore not be examined in this disclosure.

Handheld UVC devices may currently have limited use in cannabis growing, and with limited success. The disadvantages may include high labor costs, inconsistent light distribution, physical access to the plants, as well as health risks to the operators.

Another method to better administer UVC light to a crop may to pass one or more UVC lamps horizontally above a crop. Although this may solve some of the disadvantages stated regarding handheld UVC devices, this may be an ineffective way to administer UVC to crops. Firstly, many crops such as cannabis have a dense canopy that may shade the lower portions of the plants including the growing media and growing apparatuses etc. Secondly, due to the inverse square law of light, the UVC dosage at the top of the plant may be significantly higher than at locations below the canopy. Although effective UVC dosages may be present at the canopy, everything below that may receive ineffective dosages. This may allow pathogens to continue to infect the plant despite the top portion of the plants not showing any physical symptoms of the infection. If a novel system could be devised that could administer consistent UVC dosages throughout all portions of the plants, growing medium and growing apparatuses etc., this may be very advantageous.

If a novel system could be devised that could raise and lower an elongated linear style UVC light source that could penetrate into plant foliage to any desired depth, and also not harm the plants or get snagged, tangled or deflected by the plants or any other obstructions, this may enable UVC irradiation of a significant percentage of the plant foliage, stems, trunks, pots, growing medium etc.

If a novel system could be devised that could administer consistent UVC dosages throughout all portions of the plants, growing medium and growing apparatuses etc., and could also be wireless, automated and programmable wherein disinfection is done when workers are not present, this may be a significantly advantageous. UVC disinfection of pathogens is most effective during periods of darkness.

If a novel system could be devised that could administer consistent UVC dosages throughout all portions of the plants, growing medium and growing apparatuses etc., and cover a large growing area without any labor, and have a configuration that is cost effective, light weight and practical, this may be significantly advantageous.

If a novel system could be devised that could administer consistent UVC dosages throughout all portions of the plants, growing medium and growing apparatuses etc., and be able to be powered by electricity supplied to the moving system without the use of expensive and cumbersome festoon systems, this may be significantly advantageous.

The present disclosure may present various embodiments of the invention that may incorporate one or more of the novel advantageous features presented above. One such advantageous novel feature may be UVC probes that will herein be described.

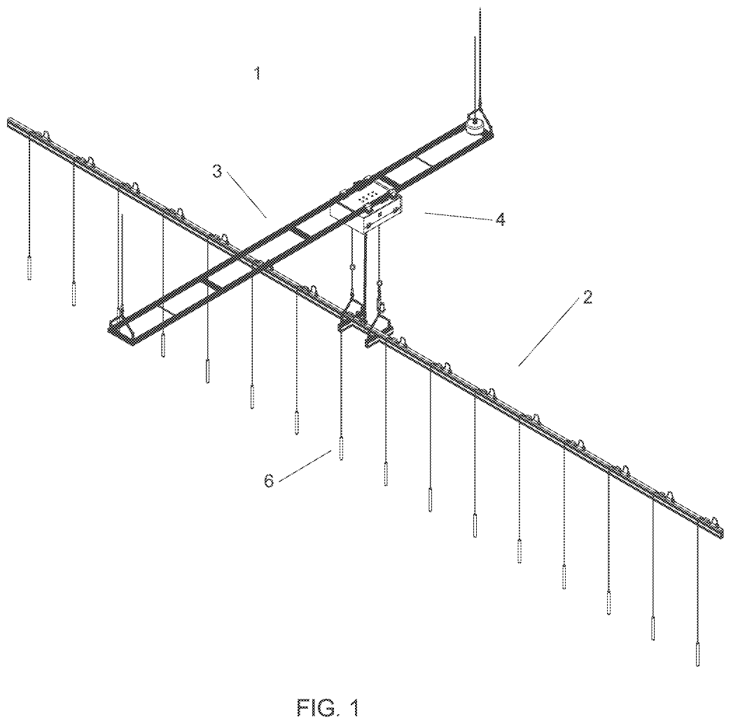

In an example embodiment shown in FIG. 1, an overview of a plant disinfection system 1 is shown, comprising a support device 2, also referred to interchangeably as a “UVC beam” with UVC probes 6 supported by the UVC beam 2, control unit 4, and track system 3. In FIG. 1 through FIG. 3 example embodiments of UVC probes may be shown in their simplest configurations for illustrative purposes.

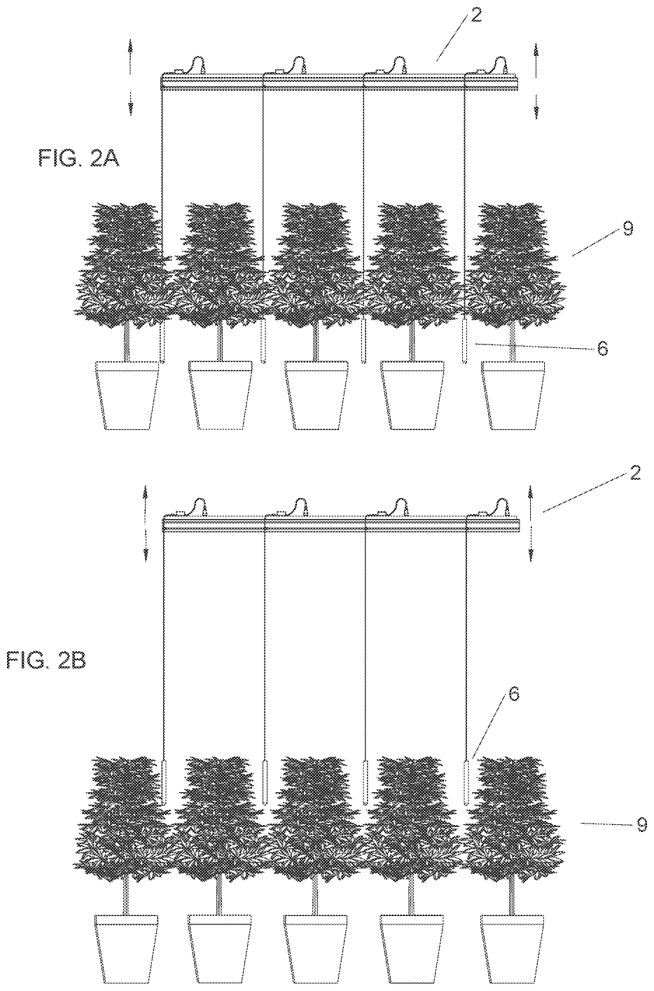

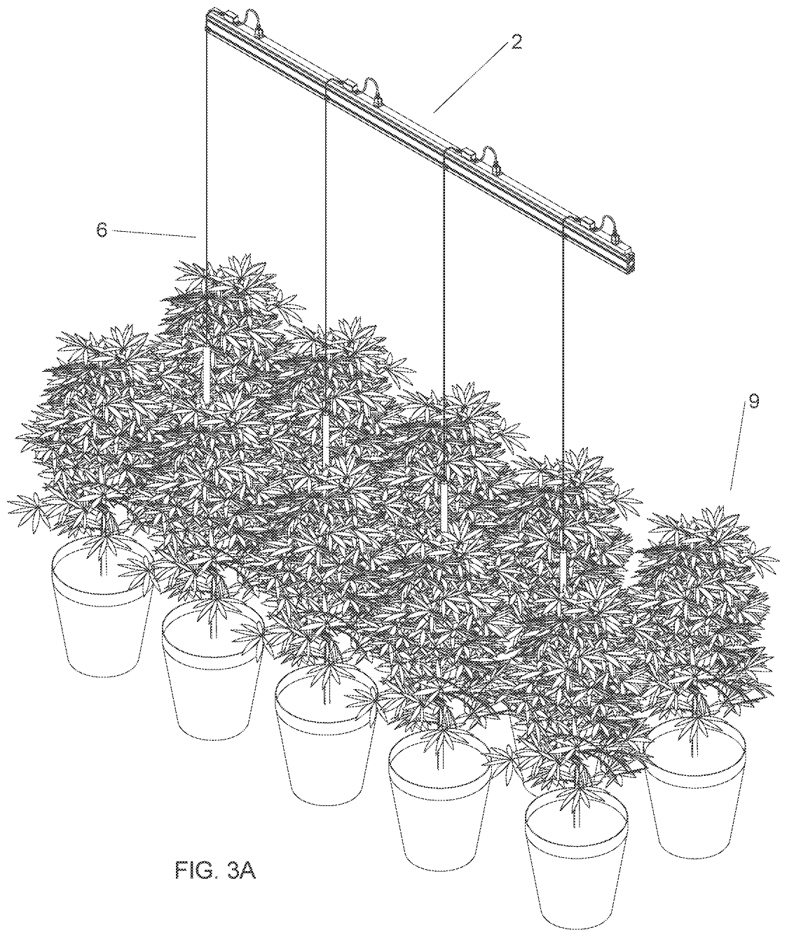

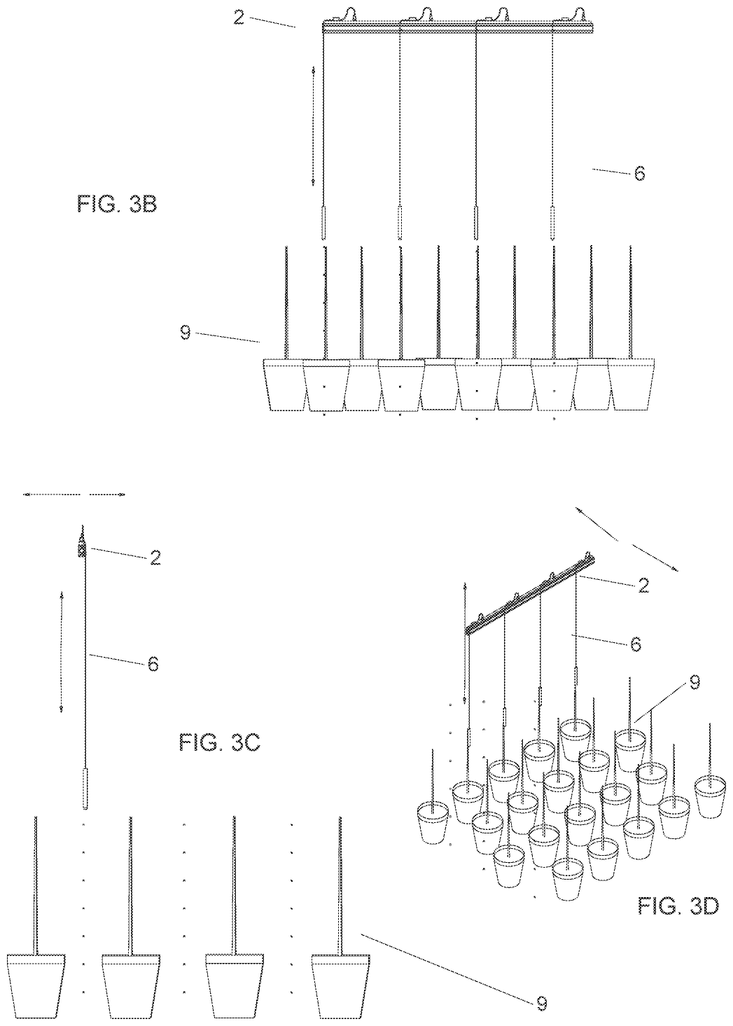

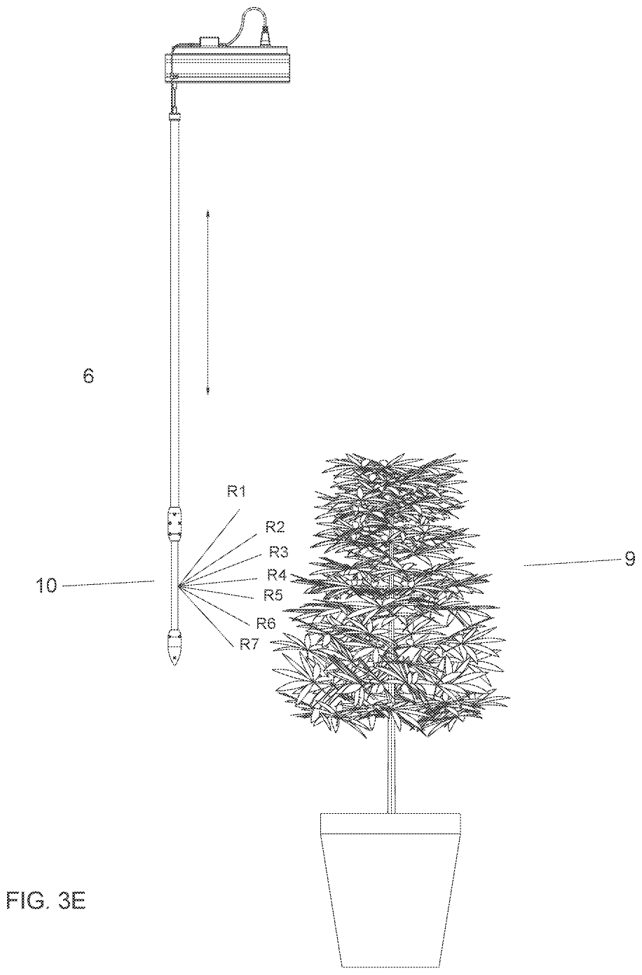

In an example embodiment shown in FIG. 2A and FIG. 2B, the UVC beam 2 may be movable in the up and down directions to any location desired below the control unit 4 (FIG. 1) as indicated by the vertical arrows. The UVC probes 6 may be lowered to a location that may irradiate the lower portions of plants 9 as shown in FIG. 2A or may irradiate upper portions of the plants 9 as shown in FIG. 2B. A perspective view of the UVC beam 2 is shown in FIG. 3A with the UVC probes 6 shown in a raised position wherein they may irradiate the upper portions of the plants 9 with UVC light.

An important element of example embodiments of novel plant disinfection systems may comprise a novel UVC probe containing a UVC light source that may be lowered and penetrate into plant foliage to a desired depth. As previously discussed, for maximum pathogen prevention and elimination, it may be necessary to distribute UVC light as evenly as possible to all parts of the plants, as well as any related surfaces such as pots, grow mediums, grow tables etc. However, real world growing conditions in greenhouse and indoor growing facilities may make any existing UVC application techniques impossible, impractical, or cost prohibitive due to hindrances such as dense thick canopies, trellis nets, poles, other obstacles or dense plant spacing. As previously discussed, a horizontally oriented UVC light source above the plants may function inadequately for several reasons. In example embodiments of UVC probes, a vertically oriented light source wherein UVC light is distributed horizontally along to the length of a plant as shown in FIG. 3E (side lighting) may have the advantage of being able to be lowered and raised into, and through plant foliage to any desired depth. When the UVC light source comprises a 360-degree light distribution characteristic such as fluorescent tubes for example, and said UVC light source is positioned on multiple sides of a plant, this may enable UVC irradiation of a significant percentage of the plant foliage, stems, trunks, pots, growing medium etc.

In FIG. 3E in an example embodiment, a UVC probe 6 comprising a UVC light source 10 (a low-pressure mercury fluorescent tube) may be positioned next to a plant 9. Example light rays R1 through R7 may emanate from a point on the UVC light source 10 and strike the plant 9 wherein UVC light is distributed horizontally along to the length of a plant (side lighting).

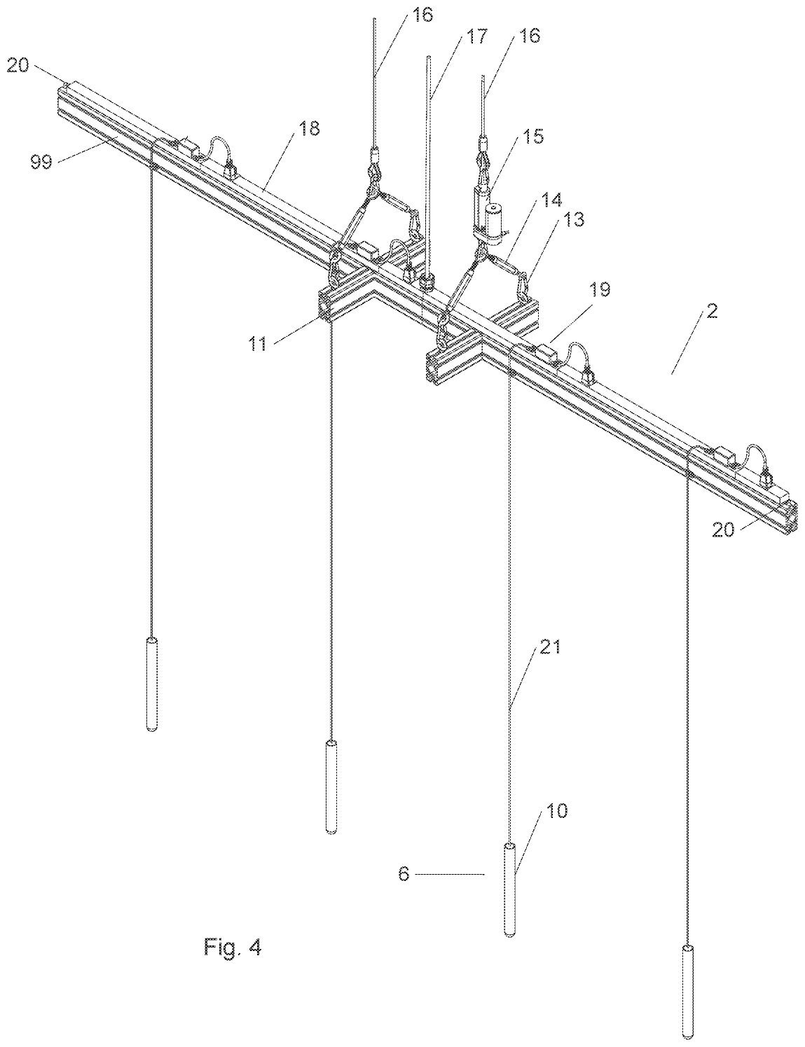

In example embodiments, UVC light sources may comprise any configuration that emits light in the UVC light spectrum (approx. 200-300 nm). For example, the UVC light sources (feature 10, FIG. 4 for example) may comprise one or more linear 254 nm low pressure fluorescent tubes, one or more compact fluorescent lamps, one or more LED arrays or lamps that emit UVC light (preferably in the 222 nm to 280 nm range), or one or more krypton chloride exclimer lamps that may emit 222 nm UVC.

In an example embodiment as shown in FIG. 3B, plants are shown without foliage for illustrative purposes only. Example embodiments of UVC probes disclosed generally may have a linear shape, a relatively small diameter, and a generally streamlined exterior without protrusions that may snag in trellis nets, branches, stems, foliage etc. In example embodiments of UVC probes, they may have a certain weight, rigidity and ability to pivot the UVC probe around any support device that may allow them to penetrate into, and through foliage to a required depth. For maximum effectiveness, having the UVC probes configurable so that they will be disposed substantially vertically in between adjacent plants may allow for the most optimal UVC light distribution.

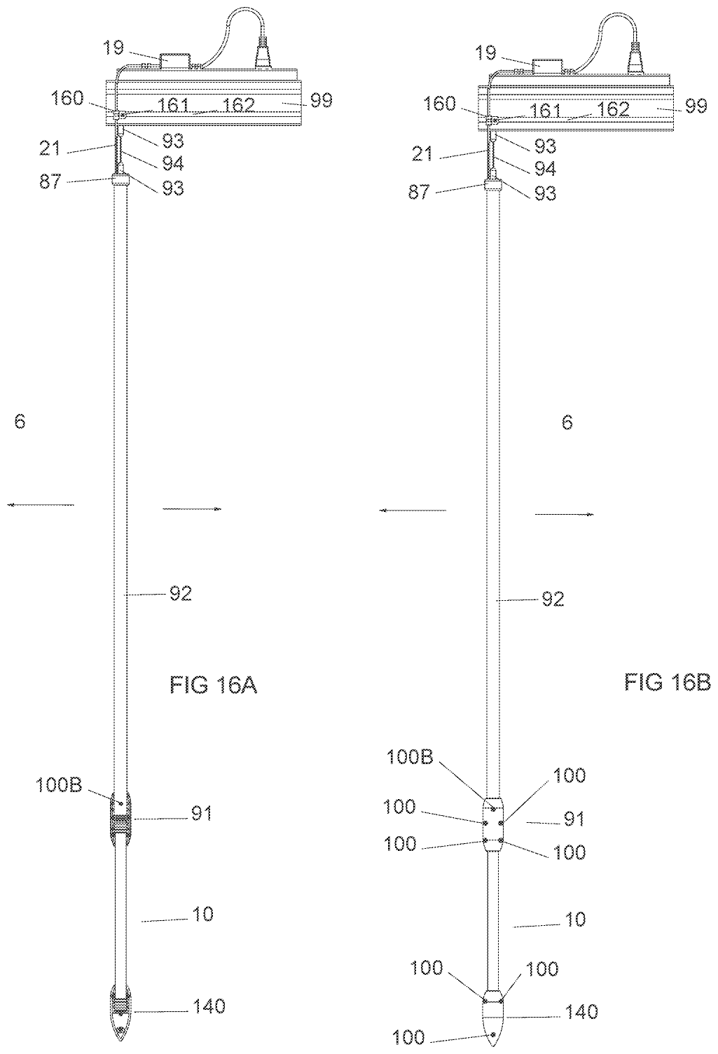

In greenhouse and indoor growing facilities, plant spacing in the growing area may be optimized to obtain the optimal number of plants in a given surface area while obtaining the highest yield per sq. ft. As such, plant spacing may be highly regular and precise. In an example embodiment as shown in FIG. 3B, a profile view in the X plane which may be parallel to the UVC beam 2 is shown. The direction of horizontal travel of the UVC beam is shown by the corresponding arrows in FIG. 3D and FIG. 3C. Due to the laterally configurable positions of the UVC probes 6 on the UVC beam 99 as shown by the arrows in FIG. 16A, the UVC probes in FIG. 3B may be configured to be in the middle of each row of adjacent plants as indicated by the dotted lines. Referring to FIG. 3C, the horizontal travel direction in the Y plane is indicated by the corresponding arrows. The middle position between adjacent rows indicated by the dotted lines may be achieved by programming the control unit 4 (FIG. 1) to stop at each desired location. As shown by the vertical arrows in FIGS. 3A, 3B and 3C, the vertical arrows indicate that the UVC probes are movable in the up/down directions, which is also programmable as previously discussed. Accordingly, the UVC probes can be positioned anywhere in the portion of a crop that is disposed beneath the span of the UVC beam 2 (FIG. 1), and the length of the track system 3 (FIG. 1). The length of track may only be limited by the reach of the power feed system (which will subsequently be discussed further), and the span of the UVC beam. Multiple plant disinfection apparatuses 1 (FIG. 1) may be utilized which may function to cover the entirety of a given growing area.

Referring to FIG. 3B through FIG. 3C, UVC beam 2 as disclosed, may allow up and down movement of the UVC probes that may allow UVC to be applied to the plants at any or all times during an up/down cycle as shown by the arrows. Since UVC dosage may be defined as UVC intensity multiplied by the time, the dosage may be controlled by the speed of the up/down cycles. In example embodiments of plant disinfection apparatuses, most, if not all parameters of the up/down cycles may be defined, programmed, and executed.

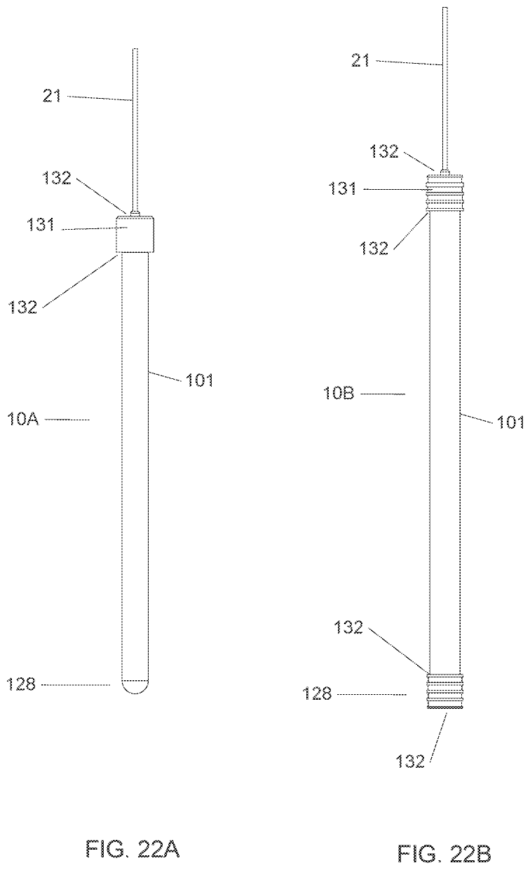

Another requirement of a UVC probe in embodiments of plant disinfection apparatuses may be that a means of suspension must be utilized that attaches directly or indirectly between the UVC light source and a support device. Out of necessity, all UVC light sources may have a power cable (or cord) attached to at least one end. This may introduce further detrimental functional elements such as hindrances 132 in FIG. 22A that may cause problems during raising and lowering of the UVC probe as described. Example embodiments of UVC probe may utilize the power cable as a suspension device if applicable. However, this may not be suitable in many applications. Electrical safety codes may not allow the suspension of a lamp with high voltages in such a manner, especially if the UVC probe may have other elements that may increase the weight of the probe. In such cases an alternate suspension device made need to be utilized which may also introduce detrimental problems as previously discussed.

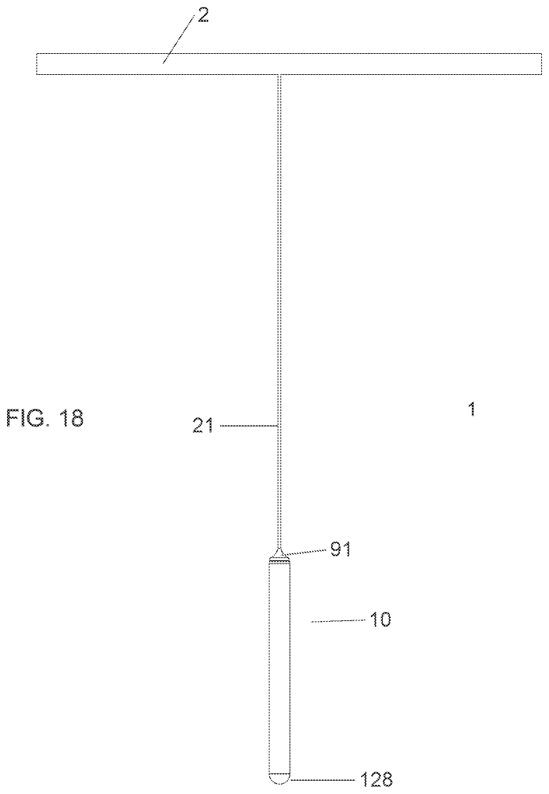

An example embodiment of UVC probe shown in FIG. 18. UVC probe 1 may comprise a light source 10 which may further comprise a power cable 21 attached to support device 2. Transition device 91 and penetration device 128 are also shown and will be discussed in further detail later in this present disclosure. The support device 2 may comprise any suitable configuration that may function adequately in a given application. An additional support wire (not shown) such as wire rope may be added in parallel to the power cable to add additional support as discussed.

In an example embodiment shown in FIG. 16A and FIG. 16B, a hollow tube 92 may function as a suspension device for UVC probe 6. A hollow tube may be configured from plastic or metal. Plastic may be preferable due to its lighter weight and resistance to moisture. For example, ½″ PVC tubing may be suitable in some example embodiments of UVC probes. The hollow tube 92 may attach to a support device (in this example embodiment UVC beam 99) using any suitable means. For example, a cap 87 may thread onto the end of hollow tube 92, and a pair of wire rope grippers 93 and a length of wire rope 94 may function to attach the hollow tube suspension device 92 to the support device 99. The opposing end of the hollow tube suspension device may attach to a transition device 91 utilizing one or more screws 100B. The hollow tube 92 may also act as a containment device to cover the power cord 21 and eliminate the power cord 21 as being a hindrance.

Other requirements of a UVC probe in embodiments of plant disinfection apparatuses may include the probe's weight, stiffness, and ability to pivot around its support device. A linear style UVC light source such as those shown in FIG. 22A and FIG. 22B may have very little weight, perhaps ½ lb., and due to the flexibility of the power cable 21, when the UVC light source 10A or 10B is lowered into foliage, there may be a high risk of deflection and ensnarement of the UVC light sources 10A or 10B by the plants. For example, the UVC light source may simply lay on stems, branches, leaves etc. when lowered into the plants. A probe with sufficient stiffness and weight in the vertical direction may be required to successfully penetrate vertically into the foliage. Due to the mobility of the support device (FIG. 16A, feature 99 for example) and the UVC probe 6, the probe may need to be capable of pivoting in the upper regions to avoid breakage of the probe and possible damage to the plants. Again, referring to FIG. 16A and FIG. 16B in an example embodiment, the hollow tube 92 may also function as the main body of the UVC probe which may add the required weight and stiffness to the UVC probe.

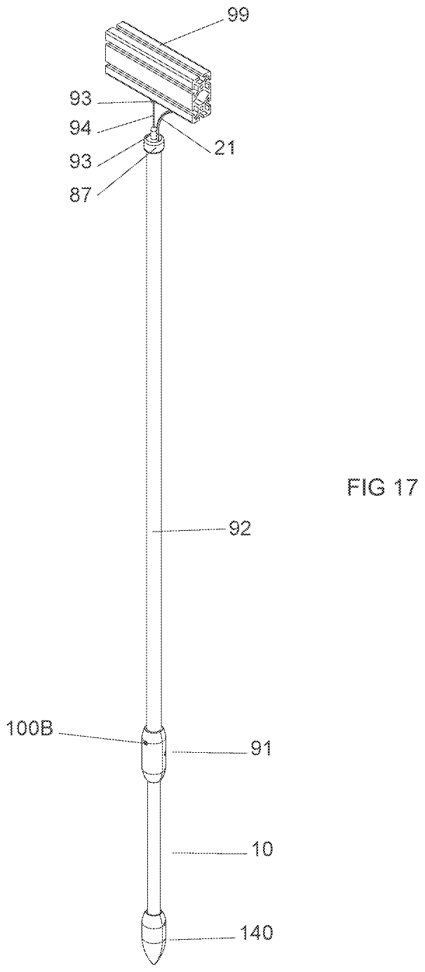

Referring to FIG. 16A, FIG. 16B and FIG. 17 in an example embodiment, the UVC probe is shown being suspended from a support device (UVC beam) 99 using a wire rope gripper 93 that may be attached to UVC beam 99. A wire rope 94 may slidingly engage, and securely attach to the wire rope gripper 93. Another gripper 93 may be attached to a probe tube cap 87, which may attach to probe tube 92. This arrangement may function as a pivot joint and may also allow quick replacement of the UVC probe. FIGS. 16A, 16B and 17 also shows example embodiments of penetration device 140, transition device 91, screw 100B to attach the transition device 91 to the hollow tube 92, and UVC light source 10.

UVC light sources may be commercially available as water submersible, such as those designed for water purification etc. Other commercially available UVC light sources may primarily be designed for air purification, and therefore may not include any water ingress protection. Submersible UVC lamps may comprise an outer quartz glass tube and gasket plug or cap 131 as shown in FIGS. 22A and 22B. Quartz glass may be able to refract UVC light without emitting significant quantities of ozone. Another advantage that water submersible UVC light sources may have may be the extra layer of quartz glass protection. Environments where example embodiments of UVC probes may be used may include obstacles such as metal poles, metal grow tables etc. which may present a breakage hazard to the glass in the UVC light sources. In the case of fluorescent light sources, broken glass may allow mercury contaminants to be dispersed into the crop. In such cases, entire areas of the crop may need to be disposed of and the areas thoroughly cleaned, which could be extremely costly.

Another important advantage of submersible UVC light sources may be exposure to moisture. Plants, especially cannabis, are grown in high relative humidity environments and may have water condensation present. Plants may also be wet from irrigation or various medicinal liquid applications. Additionally, the UVC probes may be required to be hosed down periodically for reasons of cleanliness. In example embodiments of UVC probes that may utilize fluorescent UVC light sources, the ballast voltage may high, perhaps in the range of 400V, wherein arcing between conductors or lamp pins and associated connectors in moist or wet environments may be a substantial concern.

A fundamental requirement of example embodiments of UVC probes may be not harming the plants or becoming snagged or deflected on obstructions during lowering as described elsewhere in this disclosure. Similarly, the probe must also be raised without similar problems. Elements of example embodiments of UVC probes that address this requirement may subsequently be discussed. The term “hinderance” may be used to describe a feature that may cause problems with the UVC probe's functionality, such as becoming snagged or deflected on plants or obstructions during raising or lowering, remaining substantially vertical, harming the plants or any other associated problems described elsewhere in this disclosure.

In example embodiments of UVC probes, a novel penetration device may be incorporated therein. A penetration device may comprise a bottom end of a UVC light source or UVC probe that may allow penetration into, and through plant foliage and or deflect off trellis nets or any other obstructions without becoming snagged or otherwise have its trajectory unsatisfactorily deviated from, and to allow it to remain substantially vertical, whether it is being raised or lowered. An example embodiment of a penetration device that is integral to the UVC light is shown by way of feature 128 in FIG. 22A.

FIGS. 22A and 22B shows two different variations of commercially available submersible UVC low pressure mercury lamps 10A and 10B. Both lamps may comprise a power cable 21, a top cap 131, and an outer glass tube 101. Lamp 10A may comprise a bottom end 128 that may be curved and an integral part of the outer glass tube 101, and lamp 10B may have a bottom end 128 that may comprise a cap. Example embodiment of UVC probes that have been shown with a rounded lower tip such as UVC probe lower tip 128 in FIG. 16 may function suitably as a penetration device in some example embodiments of UVC probes. In such configurations however, there may be poor protection against breakage of the glass tube, which may be the most vulnerable part of the UVC probe in example embodiments. Many submersible UVC light sources may have blunt ends or ends with hinderances or obstructions such as 128 in FIG. 22B that may otherwise cause poor penetration capabilities and may have higher chances of becoming snagged or deflected as previously described. The end cap 128 as shown may comprise a hinderance 132 between the edge of the cap 128 and the outer glass tube 101. Although relatively small, this hinderance may be configured such that it may still snag on trellis nets or may cause abrasions on foliage. The end cap 128 may also have a blunt end 132 that may be a hinderance.

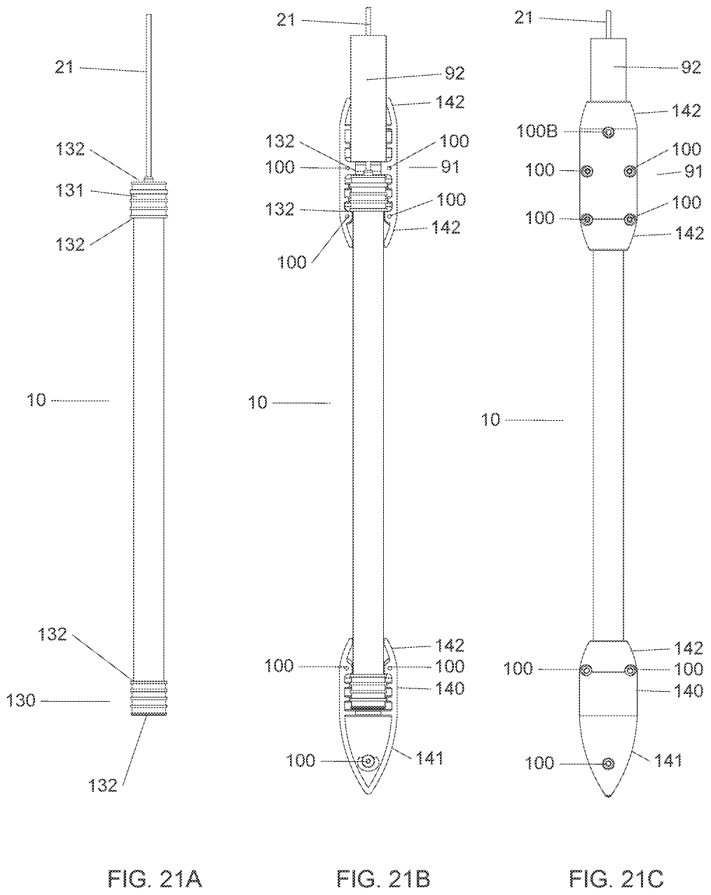

A novel device may be configured into the bottom ends of example embodiments of UVC probes which may avoid snagging, abrasions or deflection as previously described. A penetration device may comprise a bottom end of a UVC probe that may allow penetration into, and through plant foliage and or deflect off trellis nets or any other obstructions without becoming snagged or otherwise have its trajectory unsatisfactorily deviated from, and to allow it to remain substantially vertical, whether it is being raised or lowered. FIG. 21A shows a standard submersible low cost UVC light source 10 with a bottom end cap 130, an upper end cap 131, power cord 21 and hindrances 132. FIGS. 21B and 21C shows an example embodiment of a UVC probe penetration device 140 attached to the same UVC light source as shown in FIG. 21A. In example embodiments, the penetration device may be fabricated by any suitable means, such as molded plastic for example, and may comprise a penetration tip 141 and a hinderance transition feature 142. The penetration tip 141 may function to aid in the UVC probe's penetration into foliage as previously described. The hinderance transition 142 may function to bridge the transition between the glass tube and the end cap 130 to avoid snagging, abrasions or deflection as previously described. The penetration device 140 may be fabricated in two halves, wherein the two halves are fastened together with screws through screw holes 100 to clamp the penetration device 140 to the bottom cap 130 of the UVC light source 10 as shown in FIGS. 21B and 21C. Any other suitable configuration may be utilized that may produce similar advantages. Ideally, example embodiments of penetration devices should not overlap the discharge filaments inside the UVC light source 10 which may cause premature plastic degradation due to the UVC light, as well as decreasing the UVC light output. Example embodiments of penetration devices may also clamp onto an end of a UVC light source with a curved bottom end 128 in FIG. 22A for example.

The scope of possible configurations of penetration devices should not be construed to be limited by the example embodiments discussed. Different UVC light sources may have different size and shape configurations that may require different size and shape configurations of penetration devices accordingly.

Submersible UVC lamps (or any other UVC light source) may have an end that is configured to be, or is connected to a power cable such as the top end of the UVC light source 10A and 10B as shown in FIGS. 21A and 22B. The top cap 131 creates two hinderances 132, one between the outer glass tube of the light sources 10A and 10B and the top cap 131. These hinderances 132 can cause issues as previously described. A novel transition device may also be incorporated into example embodiments of UVC probes to minimize said issues. In example embodiments of UVC probes, a transition device may be used to create a smooth transition between the top of a light source and the power cable (or suspension cables if utilized) or other suspension devices such as hollow tube 92 in FIG. 16A for example.

In an example embodiment of UVC probe 1 shown in FIG. 18, any support cable (not shown) or power cables may be left exposed for a portion of their length, and the power cable and support cable near the UVC light source 10 may be wrapped with shrink tubing 91 or any other suitable covering which may also overlap with a top end of a UVC light source, thereby creating a transition device from the UVC light source top to the power and suspension cables (if included).

In an example embodiment shown in FIG. 16A, 16B, 17, 21B, 21C, a transition device 91 is shown. The transition device may create a smooth hindrance free transition between the hollow tube 92 and the top of the UVC light source 10. The transition device 91 may be fabricated from any suitable material such as plastic or metal. As shown in FIGS. 21B and 21C, the transition device 91 may be fabricated in two halves and fastened together with screws through screw holes 100. Referring to FIGS. 21B and 21C, the transition device 91 may comprise an upper and lower hindrance transition feature 142. The lower hindrance transition feature 142 may transition between the top cap of the UVC light source 10 and the outer glass tube of the light source 10, and the upper hindrance transition feature 142 may transition between the hollow tube 92 and the transition device 91.

In some applications such as crops comprising very dense foliage, additional weight of the UVC probe may be required to penetrate to the required depth and to minimize deflection of the UVC probe. In such cases the hollow tube 92 in FIG. 16A for example, may function as a ballast device. Due to the tube being hollow, any suitable material may be placed inside the tube to add additional weight, provided it does not damage or interfere with the power cable 21. For example, lead shot may be suitable.

In example embodiments, the power cable 21 in FIG. 16A from the UVC light source 10 for example, may comprise quick disconnect terminals that attach to a power cable inside the hollow tube 92. Since UVC light sources may need to be replaced approx. every 8000-10,000 hours, a replacement lamp may be quickly and easily changed out on site.

In example embodiments as shown in FIG. 4 for example, UVC light source power cord 21 attached to UVC light source 10 in UVC probe 6 may connect to a suitable ballast or LED driver 19 mounted on the support device 99, and subsequently may connect to a power distribution system 18, whereby the UVC lamp ballast 19 may be powered. Note that a similar arrangement may be utilized regardless of the configuration of example embodiment of UVC probe utilized.

In an example embodiment as shown in FIGS. 16A and 16B, the UVC probe 6 and UVC beam 99 may be configured such that the UVC probe may be laterally adjusted in the general direction of the arrows. This may be an important feature in example embodiments of plant disinfection apparatuses, as plant spacing may vary for different crops and different grow setups. When T-slot extrusions are utilized for the UVC beam 99, T-nuts (not shown) may slidingly engage with the T-slots 162, allowing them to be slid into position and tightened where needed. Cable clips 160 may be attached to the T-nuts with screws 161 wherein the cable clips may secure the power cord 21 which may connect to ballast or LED driver 19.

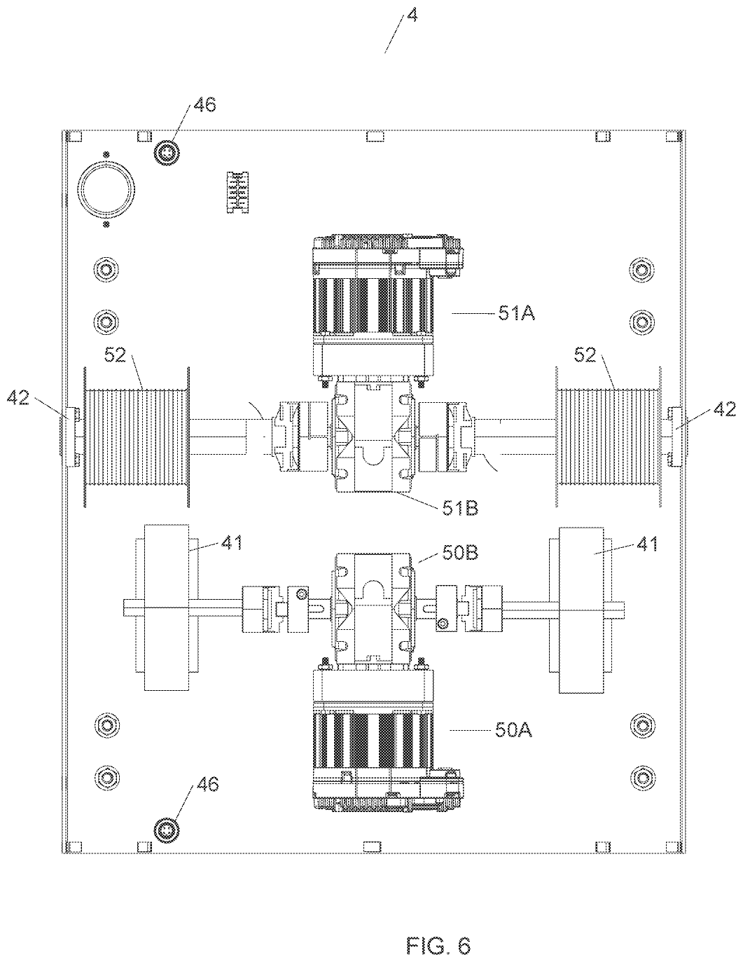

In an example embodiment as shown in FIG. 4, a detailed view of a UVC beam 2 is shown. A main beam 99 may be configured as the main structural component, which may comprise any suitable material or configuration. For example, as shown, so called “T-slot” aluminum extrusions may be utilized, which may have the advantage of being off the shelf items that comprise a very high strength to weight ratio. As shown, the main beam 99 may comprise a weight of 2.42 lbs. per foot and incur a total deflection of less than one inch over a 32′ span. The main beam 99 may also comprise cross beams 11 for added balance and support of the UVC beam 2. Hanger assemblies are shown attached to the cross beams 11 which may comprise aircraft wire rope 16 that may windingly attach to take-up spools (feature 52 in FIG. 6), turnbuckles 14, and carabiner clips 13. Power cable 17 may supply power from the control unit (not shown) to the power distribution system 18. A linear actuator 15 is shown connected in series with a wire rope 16. Hanger assemblies may also comprise any suitable format or configuration that may impart the desired strength, balance and adjustability required for a given application.

In an example embodiment as shown in FIG. 4, a linear actuator 15 may be included in-line with one or both of the hanger assemblies, whereby tilt sensors 20 may communicate positioning information to associated hardware in the control unit 4 (FIG. 1) that may correct any UVC beam 2 deviations from the horizontal plane in real time. This novel feature may be important so that the UVC beam 2 does not contact the plants or as an emergency stop if a UVC probe becomes snagged.

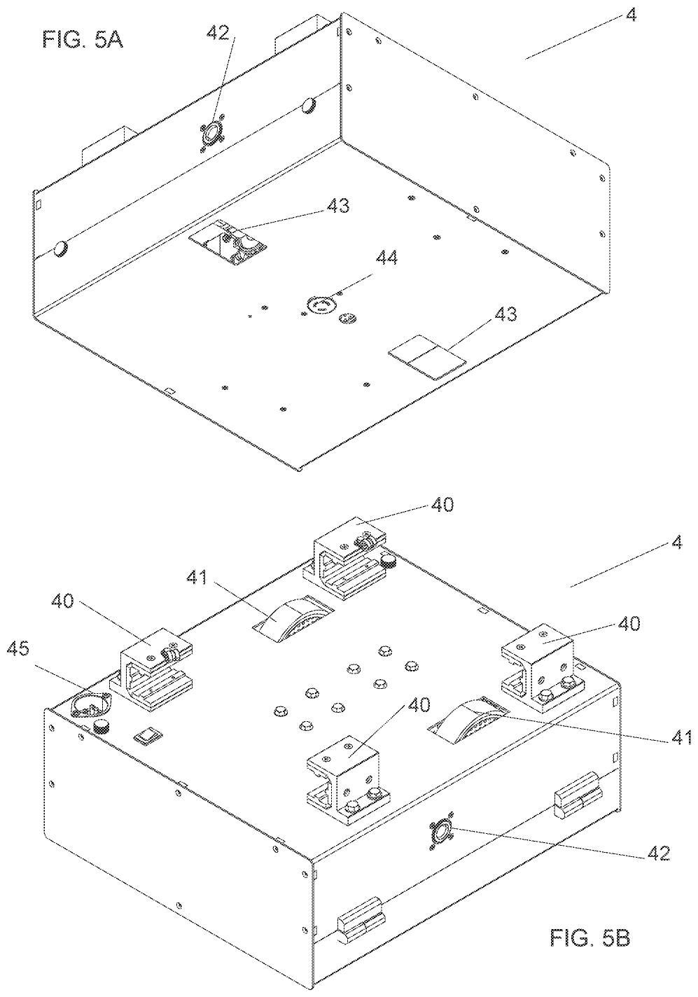

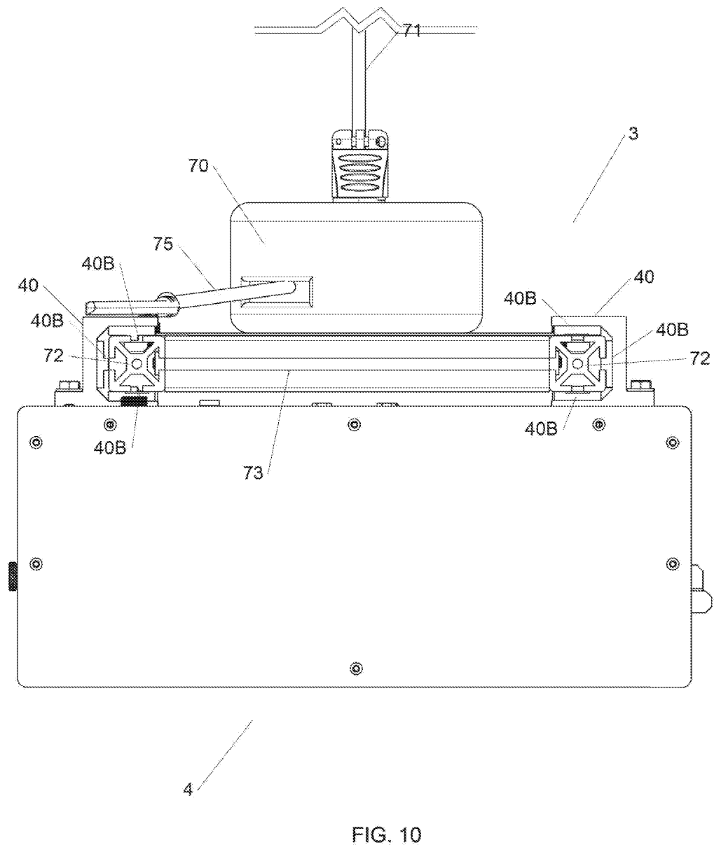

In an example embodiment, FIG. 5A shows an underneath perspective view of the control unit 4, and FIG. 5B shows a top perspective view. As shown in FIG. 5B, sliding blocks 40 may comprise aluminum U-shaped configurations with low friction and self-lubricating plastic linings that may slidingly engage with track rails 72 (FIG. 8). Other configurations of sliding devices may also be utilized. For example, assemblies with wheels that engage with the track rails 72. Drive wheels 41 (also shown in FIG. 6) may be controlled by one or more motors 50A with associated gearbox 50B (FIG. 6) that may engage with the track rails 72 (FIG. 8) to move the control unit 4 along the tack system 3 (FIG. 1). FIG. 10 shows a partially cut-away profile view showing the sliding blocks 40 with plastic linings 40B slidingly engaged with track rails 72. Power cable 71 may provide power to the cable reel 70. The track system 3 is also shown. Support bearing 42 for the take up spools (52, FIG. 6, FIG. 5A, FIG. 5B) are utilized to add axial and radial support.

In an example embodiment as shown in FIG. 5B, power to the control unit 4 may be supplied by a novel powering system that may connect to power receptacle 45. This novel system will be discussed in more detail later in this application. Stop sensors 46 in FIG. 6 may be utilized at both ends of the control unit 4 to stop the moving unit at desired locations.

In an example embodiment, FIG. 5A shows lift wire openings 43 wherein the lift wires 16 (FIG. 4) may enter the control unit 4 and windingly attach to take up spools 52 (FIG. 6). Power receptacle 44 may supply power to the UVC power cable 17 (FIG. 4).

In an example embodiment, FIG. 6 shows an internal view looking up into the control unit 4. Drive motor 50a and associated gearbox 50B attach with drive couplings to drive wheels 41. A similar lift motor assembly 51A and 51B may be used to drive the take up reels 52. Stop sensors 46 are shown. The motors may comprise any type of suitable motors, such as DC stepper motors for example. It may be preferable for the motors to have feedback capability to enable a computer to control the motors.

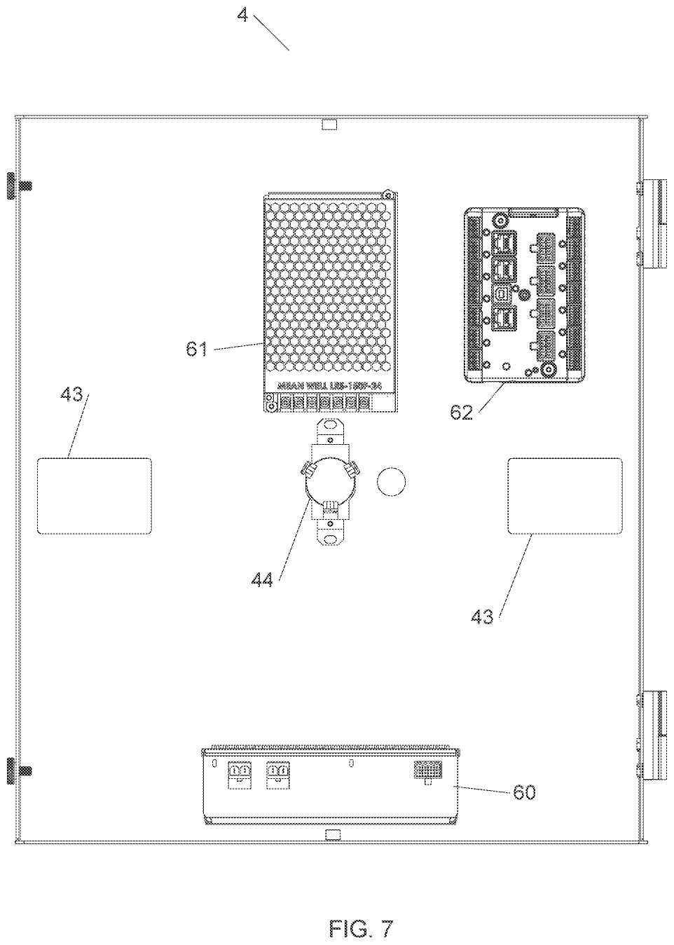

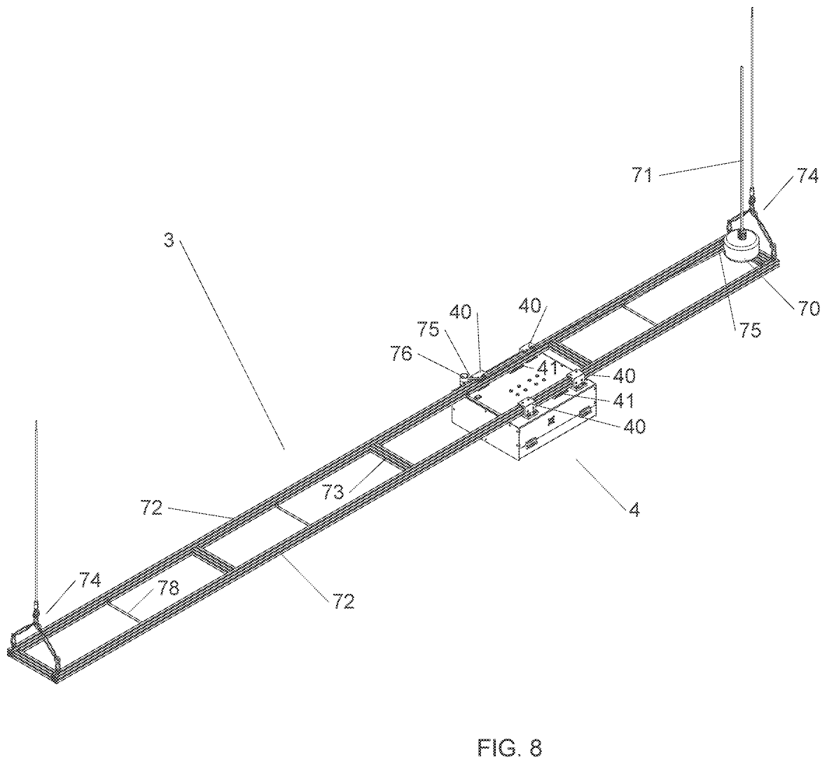

In an example embodiment, FIG. 7 shows an internal view of the electronic bay of the control unit 4. A power supply 60 may power both the drive motors and lift motors, and a power supply 61 may power a computer module 62. Lift wire openings 43 and UVC power receptacle 44 are also shown.

In an example embodiment, the computer module may comprise any suitable configuration that may be able to operate the control unit 4 as needed. Off the shelf motor control computers can be sourced at acceptable prices. Customized software for the computer module may be configured to add the desired functionality to example embodiments of plant disinfection systems. For example, preferable programmable features may include any variations of control unit movements along the track system 3 (FIG. 1) and up/down movements of the UVC beam 2 (FIG. 1). Real time control features, timer features, safety shutdown protocols etc. may be included in example embodiments as well. Wireless control of example embodiments of plant disinfections apparatuses may be significantly advantageous since the apparatuses may be suspended above ground and relatively inaccessible.

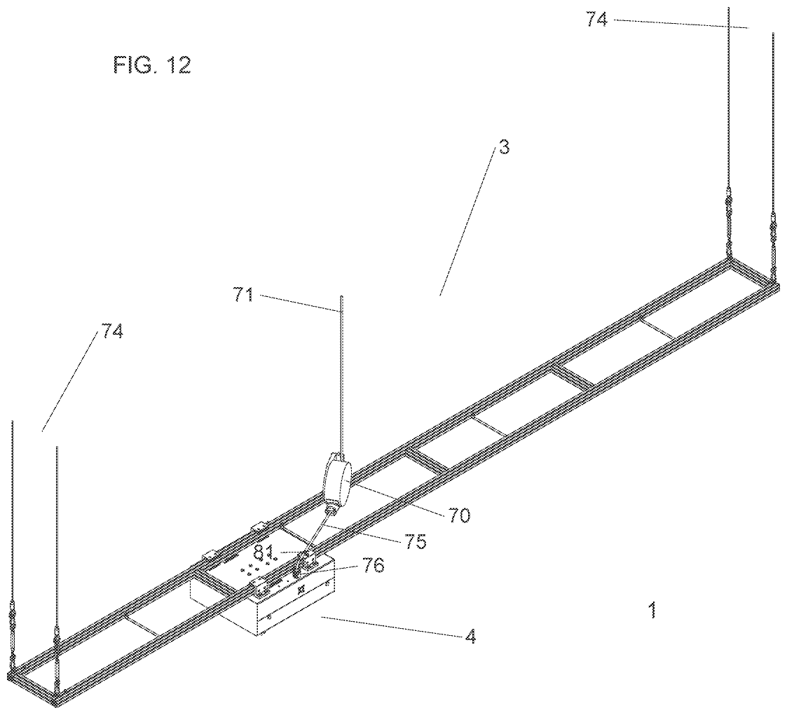

In an example embodiment, FIG. 8 shows a detailed view of the track system 3. The track system 3 may be hung in a similar fashion as with the UVC beam with hanging systems 74. The hanging system 74 may be configured as shown, or may comprise two suspension wires/points as shown in FIG. 12, feature 74. Referring to FIG. 8, sliding blocks 40 may slidingly engage with track rails 72, propelled by drive wheels 41. Track support crossbeams 73 may give the track system addition rigidity. A main power feed 71 may supply power from the building's power into cord reel 70. Retractable power feed cable 75 may supply power to the control unit 4 through plug 76. Straps 78 may be utilized to help minimize power cable 75 droop.

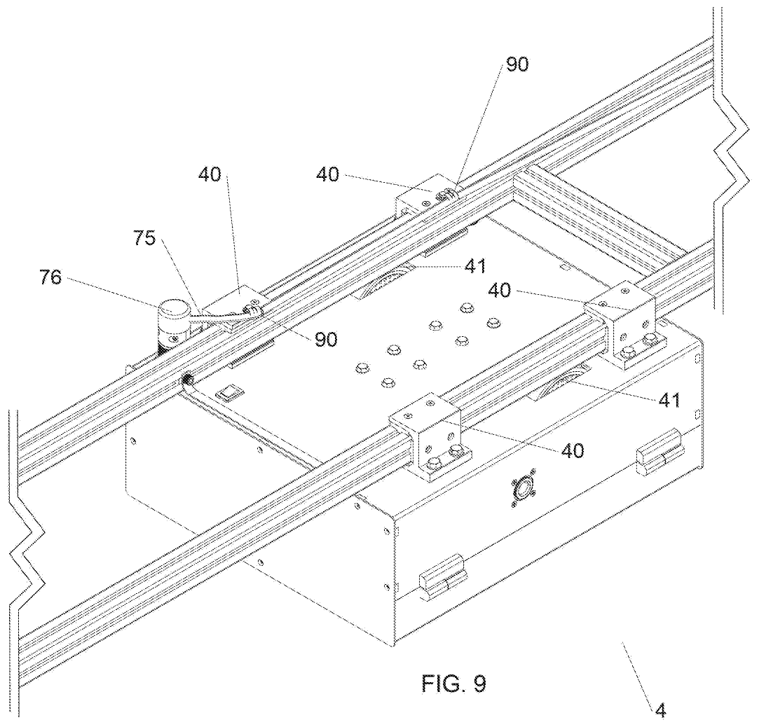

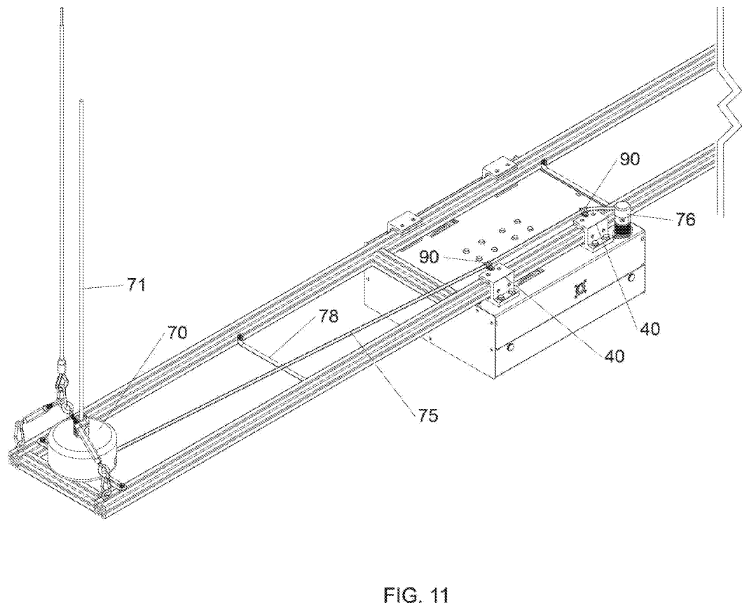

In an example embodiment, details of novel power supply systems for moving apparatuses are shown and discussed. Typically festoon systems may comprise multiple loops of cable wherein each loop may be hung on an individual trolley with wheels, wherein the wheels slidingly engage with a track system. This type of system may be expensive and have multiple long loops of cable hanging below the track which may both be undesirable attributes with regards to expense, practicalities in a grow environment, aesthetics etc. For example, a festoon system may require its own track system that would significantly increase the weight and expense of an example embodiment of plant disinfection system. In an example embodiment of a retractable power cable apparatus, and collectively referring to FIG. 9 through FIG. 11, cord reel 70 may comprise a typical spring-loaded retractable cable reel wherein the latching feature is removed, thereby creating constant tension on the power cable. The power cable 75 (the power output cable from the cable reel) may be securely attached to the sliding blocks 40 utilizing cable clamps 90. The plug 76 may be mated with the corresponding receptacle 45 (FIG. 5B). As the control unit 4 (the moving apparatus) moves along the track, power cable 75 may be released and retracted under constant tension and on a relatively direct pathway between the control unit 4 and the cord reel 70. The power cable may lay disposed on cross bars 73 and cable brackets 78 FIG. 11) as the control unit 4 advances away and towards the cord reel 70. This novel retractable power cable apparatus may keep retain the power cable 75 in a relatively straight orientation and disposed on a relatively direct path to the control unit 4 along the track whereby it does not become snagged or tangled.

In an example embodiment as shown in FIG. 12 through FIG. 15, the details of another retractable power cable apparatus for moving systems are disclosed. Referring to FIG. 12 in an example embodiment, pivoting cable reel 70 may mount in the middle region of the track system 3 and may supply power cable 75 to the control unit 4 from either side. Pivoting cable clip 81 attached to the control unit 4 may attach to plug 76. Power cable 71 may supply power to the cable reel 70.

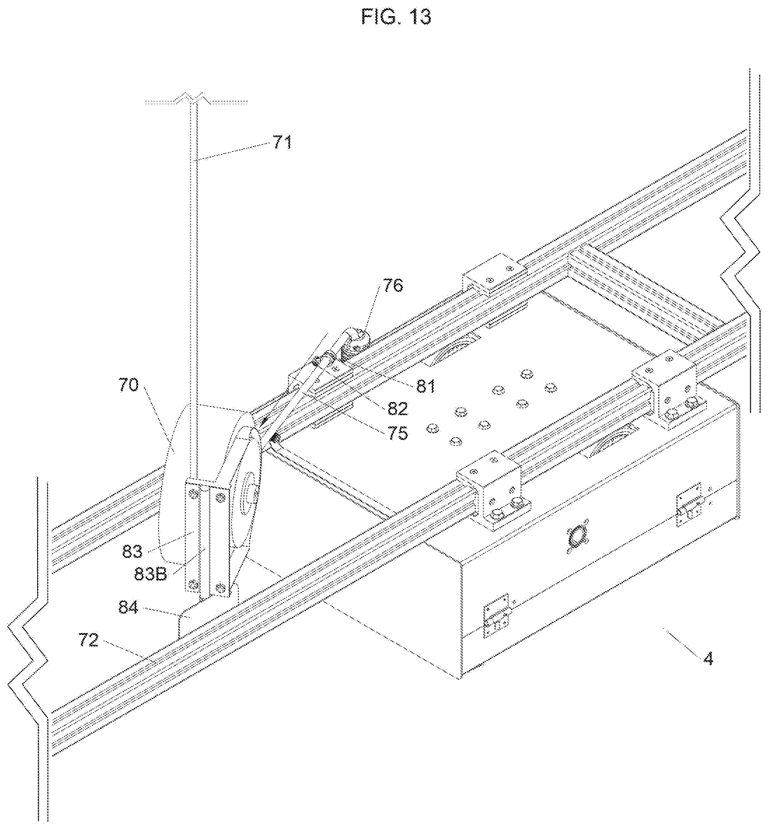

FIG. 13 shows a closer view of the example embodiment shown in FIG. 12. Pivoting cable reel 70 with power supplied by main power feed 71 may comprise a mounting bracket 83 comprising a male rod or female round receptacle 83B that mates with the corresponding counterpart on mounting base 84 which may attach to a track system rail 72 (or any other suitable fixed attachment point). Pivoting cable clip 81 may attach to the control unit 4 utilizing a thrust bearing 82. This system may allow the control unit to be fed with power from the same cable reel at any location on the track system. As a result, in an example embodiment, a single cable reel may supply both halves of the plant disinfection system, which may effectively halve the amount of electrical cable housed inside the cable reel 70. Due to the physical size and weight of large lengths of electrical cable, there may be limits on the length of cable that can be used in a cable reel. Accordingly, at whatever that limit may be, having the cable reel service both halves of a plant disinfection system may effectively double the possible useable length thereof.

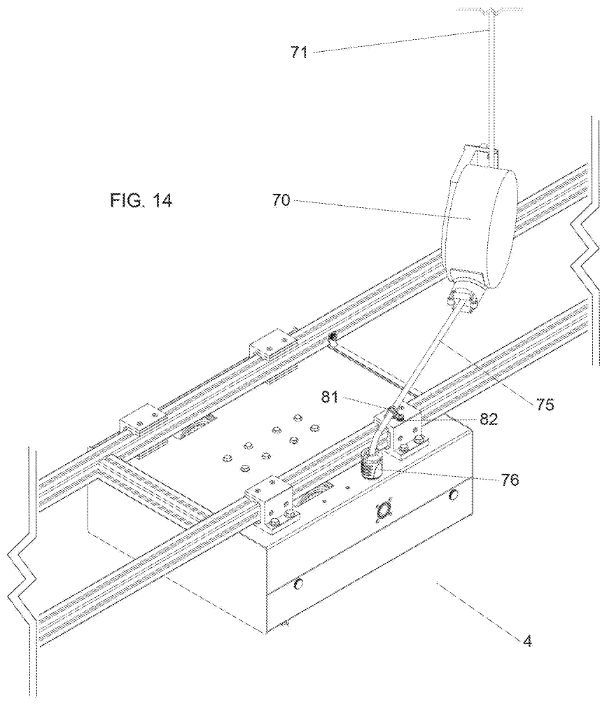

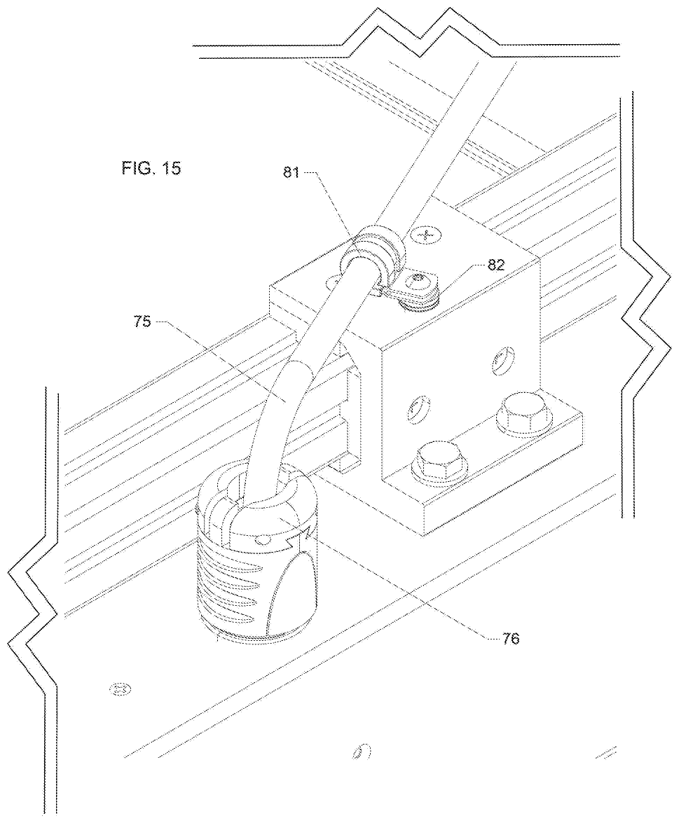

FIG. 14 and FIG. 15 show different views of the example embodiment shown in FIG. 13 with the corresponding features indicated.

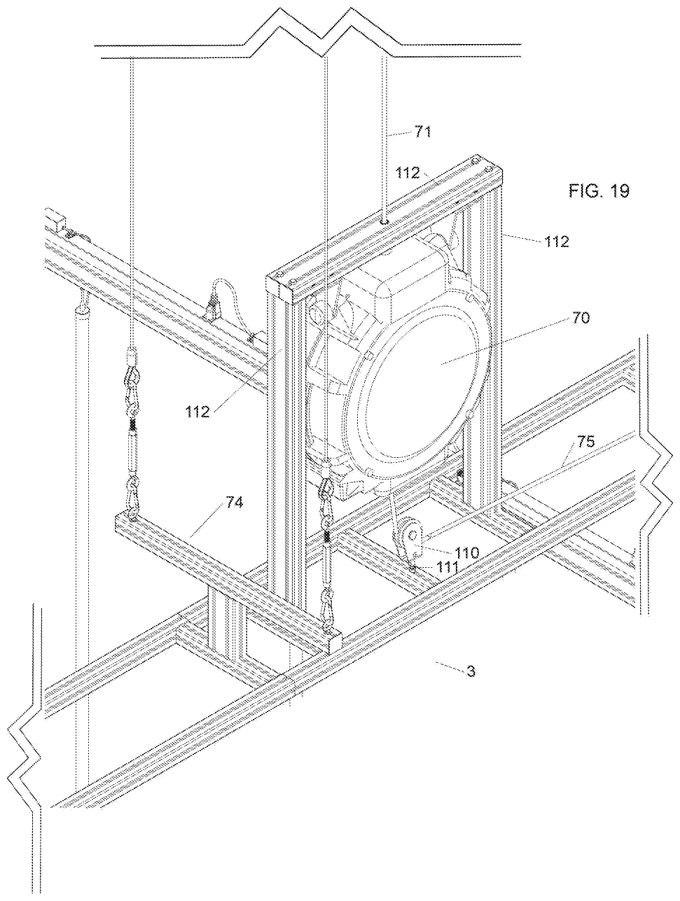

In an example embodiment as shown in FIG. 19, the details of another novel retractable power cable apparatus for moving systems are disclosed. Retractable cable reel 70 may be held in a fixed position by support members 112. The power cable 75 may be routed through a pulley 110 that may be mounted to the track system 3 utilizing a pivoting swivel ball type mount 111, that may allow the pully to rotate in two planes which may allow the power output cable 75 to retain a relatively straight path to the control unit 4 (not shown) and to minimize the degree of bending angle of the power output cable 75. The cable reel 70 may mount in the middle region of the track system 3 and may supply power cable 75 to the control unit 4 (not shown) from either side. A pivoting cable clip assembly 81 and 82 attached to the control unit 4 may attach to plug 76 (FIG. 14). Power may be supplied to the cable reel 70 by power cable 71. Another example embodiment of hanging system 74 is shown which may allow more room for the power cable 75 to be supported on. Said example embodiment utilizes a raised cross bar configuration, the details of which may need not be explained due to obviousness. Said example embodiment of hanging system 74 may be utilized on any or all example embodiments of plant disinfection device.

FIG. 18 shows an example embodiment of plant disinfection apparatus. UVC light source 10 can comprise any configuration previously discussed, and one or more UVC light sources may be utilized. The UVC light source 10 may be powered through power cable 21. Optional support wire 94 is shown, which can be used in applications where the power cable 21 cannot support the weight of the UVC light source 10 or is otherwise prohibited to do so by electrical codes. UVC beam 2 may comprise any configuration that may be suitable to suspend the UVC light source above the ground. For example, a hollow tube or rod may be used to suspend the UVC light source above the ground. The UVC beam 2 may be supported by any suitable means, such as handheld, or by any suitable mechanical means. In an example embodiment, the UVC light source may be mounted on existing horticultural equipment such as spraying and irrigation systems that are already configured to suspend mechanical devices above plants. In example embodiments, the UVC light sources may be powered by one or more batteries or may be solar powered with or without batteries.

It should be noted that the term “UVC beam” or the word “beam” may imply any elongated mechanical part or apparatus that may be utilized as a suspension device to suspend or attach to any UVC light source or UVC probes as described, envisioned, or taught in this application, and should not be construed to limit the scope of example embodiments of plant disinfection apparatuses.

In an example embodiment of plant disinfection apparatus with a similar configuration as shown in FIG. 1, the details of which have been previously disclosed, may be utilized wherein instead of a track system 3, the UVC beam may be mounted on a centrally located post wherein the beam spins around the post, thereby eliminating a track system, which may have cost savings in some applications. The post may include a means of vertical up/down movement to raise and lower the UVC beam. For example, the post may telescope or be fixed, and may move by means of electric motors, servo motors, linear actuators, hydraulics etc. All elements of the plant disinfection apparatus may be controlled and programmed for autonomous use as previously described. In an example embodiment, the track system 3 may not be not hung from a ceiling, but supported on each end by vertical supports attached to a motorized conveyor system with wheels that runs on the ground.

In an example embodiment of plant disinfection apparatus similar to that shown in FIG. 1, the control unit 4 and UVC beam 2 may be battery powered. In an example embodiment the batteries may be charged utilizing solar panels.

In an example embodiment of plant disinfection apparatus similar to that shown in FIG. 1, the control unit 4 and UVC beam 2 may be battery powered and may comprise solar panels to charge the batteries. Indoor growing applications for cannabis have very high light intensity levels, and solar panels mounted in locations that do not shade the plants may be utilized, such as near the ends of the track system 3. A relatively small surface area of solar panels may only be required to power the entire apparatus.

In an example embodiment, the UVC beam may comprise a means of raising and lowering the UVC light assemblies up and down relative to the beam. For example, a winch apparatus mounted on the UVC beam may attach to a cable and pulley system that raises and lowers the individual UVC light assemblies. The UVC beam may or may not be able to be raised or lowered.

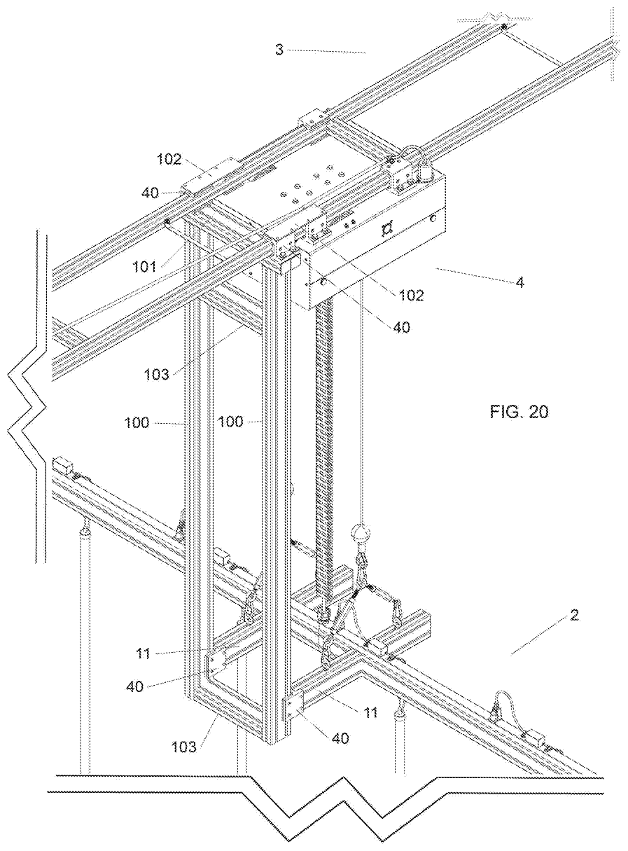

An example embodiment of an anti-sway apparatus is shown in FIG. 20. Sliding blocks 40 at the top may slidingly engage with the track system 3 and may attach to a base 101 which may support vertical frame members 100 and cross bar 103. Base 101, the vertical frame members 100 and cross bar 103 may be fabricated from T-slot aluminum extrusions as previously described or may be fabricated from any other suitable materials. Brackets 102 may securely attach the anti-sway apparatus to the control unit 4 which may allow both the control unit 4 and the anti-sway apparatus to move as a single unit. Sliding blocks 40 at the bottom may attach to cross beams 11 of the UVC beam 2 and slidingly engage with the vertical frame members 100. As the UVC beam raises, the vertical frame members may protrude through the bottom sliding blocks 40. Example embodiments of anti-sway apparatuses may function to minimize any horizontal spin of the UVC beam 2 as well as any tilting thereof.

In an example apparatus, the sliding blocks 40 made be substituted for wheels or rollers that rollingly engage with the vertical frame members 100.

In an example embodiment, an anti-sway apparatus as described may be configured on both sides of the control unit 4.

In an example embodiment of the disclosed technology, a plant disinfection apparatus is provided that may comprise a support device configured to directly or indirectly support one or more UVC probes beneath the support device. The one or more UVC probes may each comprise a relatively elongated shape comprising a major axis Y, a light source that emits UVC light in a predominantly perpendicular distribution pattern relative to axis Y, and a suspension device configured to directly or indirectly attach to the support device. The UVC probe may be configured to be raised and lowered in a relatively vertical orientation and may further comprise a means of conveying power to the UVC light source. The one or more UVC probes may be configured to be lowered into the foliage of one or more plants disposed beneath the plant disinfection apparatus.

In an example embodiment, the UVC light source may comprise one or more 254 nm fluorescent lamps, one or more LED lamps capable of emitting light in the UVC frequency spectrum, one or more lamps capable of emitting light in the approximate 222 nm light spectrum, and one or more of any other light sources capable of emitting light in the UVC frequency spectrum.

In an example embodiment, the one or more UVC probes may further comprise a transition device attached around the top of the UVC light source and at least a portion of the suspension device, wherein the transition device may be configured to provide a relatively smooth streamlined transition between the suspension device and the top of the UVC light source, thereby minimizing any hinderances that may snag, obstruct or deflect the one or more UVC probes on any plant parts, trellis nets, poles or other obstructions in a growing area.

In an example embodiment, the one or more UVC probes may further comprise a transition device attached around the top of the UVC light source and at least a portion of the suspension device, wherein the transition device may be configured to provide a relatively smooth streamlined transition between the suspension device and the top of the UVC light source, and additionally to provide a relatively smooth streamlined transition between the transition device and the outer surface of the portion of the UVC light source located below the top thereof. This may minimize any hinderances that may snag, obstruct or deflect the one or more UVC probes on any plant parts, trellis nets, poles or other obstructions in a growing area.

In an example embodiment, the one or more UVC probes may further comprise a penetration device attached to the lower tip of the UVC light sources, wherein the penetration device may comprise a curved or cone shaped lower tip to aid the UVC probe in penetrating into plant foliage in a substantially vertical orientation without causing damage to the plants, and thereby minimizing any hinderances that may snag, obstruct or deflect the one or more UVC probes on any plant parts, trellis nets, poles or other obstructions in a growing area.

In an example embodiment, the one or more UVC probes may further comprise a penetration device attached to the lower tip of the UVC light sources, wherein the penetration device may comprise a curved or cone shaped lower tip to aid the UVC probe in penetrating into plant foliage in a substantially vertical orientation without causing damage to the plants. It may further may comprise an upper transition surface on the top portion of the penetration device to create a relatively smooth and streamlined transition between the upper portion of the penetration device and the UVC light source.

In an example embodiment, the suspension device of a UVC probe may be an elongated hollow tube.

In an example embodiment, the means of conveying power to the one or more UVC probes may be a power cord that attaches to the UVC light source, wherein the power cord may be the suspension device.

In an example embodiment, the means of conveying power to the one or more UVC probes may be a power cord that attaches to the UVC light source, wherein the power cord may be the suspension device and may further comprise a transition device attached around the top of the UVC light source and at least a portion of the power cord.

In an example embodiment, the elongated support device may comprise an elongated beam configured to support two or more UVC probes, wherein the elongated beam may be configured to be raised and lowered such that the one or more UVC probes are raised or lowered into the foliage of the one or more plants.

In an example embodiment, the elongated support device may comprise an elongated beam configured to support two or more UVC probes, wherein the elongated beam may be configured to be raised and lowered by one or more cables that are attached to a control unit, wherein the control unit may be a motorized apparatus configured to move along a track system configured to be disposed above the one or more plants, such that the elongated beam can be raised and lowered along the path of the track system.

In an example embodiment of the disclosed technology, the elongated support device may comprise an elongated beam configured to support two or more UVC probes, wherein the elongated beam may be configured to be raised and lowered by one or more cables that are attached to a control unit. The control unit may comprise a motorized apparatus configured to move along a track system, a mounting system configured to securely suspend and slidingly engage the control unit with the track system, a computer controllable motor attached to drive wheels, wherein the drive wheels engage the track system to allow the control unit to move along the track system. A computer controllable motor may attach to the one or more cable winding systems wherein the one or more cable winding systems may be configured to engage the one or more cables wherein the elongated beam can be raised or lowered therein.

In an example embodiment, the elongated support device may comprise an elongated beam configured to support two or more UVC probes, wherein the elongated beam may be configured to be raised and lowered by one or more cables that are attached to a control unit. The control unit may comprise a motorized apparatus configured to move along a track system, a mounting system configured to securely suspend and slidingly engage the control unit with the track system, a computer controllable motor attached to drive wheels, wherein the drive wheels may engage the track system to allow the control unit to move along the track system, and a computer controllable motor attached to the one or more cable windings systems wherein the one or more cable winding systems may be configured to engage the one or more cables wherein the elongated beam can be raised or lowered therein. A retractable power cord system may comprise a power cable configured to be attached to the control unit, and may further comprise a tensioned reel apparatus comprising a motorized or spring tensioned reel configured to the attach to, and pull the power cable into the tensioned reel apparatus wherein the power cable may be wound around the reel and tension may be imparted on the power cable such that the power cable maintains tension between the retractable power cord system and the control unit as the control unit moves away and towards the retractable power cord system.

In an example embodiment, the elongated support device may comprise an elongated beam configured to support two or more UVC probes, wherein the elongated beam may be configured to be raised and lowered by one or more cables that are attached to a control unit. The control unit may comprise a motorized apparatus configured to move along a track system, a mounting system configured to securely suspend and slidingly engage the control unit with the track system, a computer controllable motor attached to drive wheels, wherein the drive wheels may engage the track system to allow the control unit to move along the track system, and a computer controllable motor attached to the one or more cable winding systems wherein the one or more cable winding systems may be configured to engage the one or more cables wherein the elongated beam can be raised or lowered therein. One or more anti-sway devices may be disposed adjacent to the control unit and may also slidingly or rollingly engage with the track system, wherein the one or more anti-sway devices may comprise vertical tracks that extend from the track system to the elongated beam, wherein the elongated beam may further comprise wheels or sliders that slidingly or rollingly engage with the vertical tracks such that the up and down movement of the elongated beam may be stabilized by the vertical tracks.

In an example embodiment of the disclosed technology, a power feed system to supply electrical power to a moving apparatus is provided. The power feed system may comprise a retractable power cable apparatus comprising an output power cable configured to be attached to a moving apparatus, and the retractable power cable apparatus may comprise a tensioned reel apparatus with a motorized or spring tensioned reel configured to the attach to, and pull the output power cable into the tensioned reel apparatus and wind the output power cable around a reel, wherein tension is imparted on the output power cable. The output power cable may maintain tension between the retractable power cable apparatus and the moving apparatus as the moving apparatus moves away and towards the retractable power cord system.

In an example embodiment, the retractable power cable apparatus may be disposed in a fixed location and may further comprise one side with a port that allows the output power cable to enter the retractable power cable apparatus located on that side, and wherein the moving apparatus may be configured to move in a direction away and towards that side of the retractable power cable apparatus wherein the output power cable may retain a substantially direct pathway between the retractable power cable apparatus and the attachment point on the moving apparatus.

In an example embodiment, the retractable power cable apparatus may be configured to pivot around at least one axis and may comprise a port that allows the output power cable to enter the retractable power cable apparatus, wherein the output power cable may be configured to move in a direction along the pivot axis, and wherein the output power cable retains a substantially direct pathway between the retractable power cable apparatus and the attachment point on the moving apparatus.

I claim:

1. A plant disinfection apparatus comprising:
a support device configured to directly or indirectly support one or more UVC probes beneath the support device;
one or more UVC probes each comprising:
a relatively elongated shape comprising a major axis Y;
a light source that emits UVC light in a predominantly perpendicular distribution pattern relative to axis Y,
a suspension device configured to directly or indirectly attach to the support device wherein the UVC probe is configured to be raised and lowered in a relatively vertical orientation;
a means of conveying power to the UVC light source; and
wherein the one or more UVC probes are configured to be lowered into the foliage of one or more plants disposed beneath the plant disinfection apparatus.

2. The UVC light source of claim 1 comprises one or more 254 nm fluorescent lamps, one or more LED lamps capable of emitting light in the UVC frequency spectrum, one or more lamps capable of emitting light in the 222 nm light spectrum, and one or more of any other light sources capable of emitting light in the UVC frequency spectrum.

3. The one or more UVC probes of claim 1 further comprise a transition device attached around the top of the UVC light source and at least a portion of the suspension device, wherein the transition device is configured to provide a relatively smooth streamlined transition between the suspension device and the top of the UVC light source, thereby minimizing any hinderances that may snag, obstruct or deflect the one or more UVC probes on any plant parts, trellis nets, poles or other obstructions in a growing area.

4. The one or more UVC probes of claim 1 further comprise a transition device attached around the top of the UVC light source and at least a portion of the suspension device, wherein the transition device is configured to provide a relatively smooth streamlined transition between the suspension device and the top of the UVC light source and additionally to provide a relatively smooth streamlined transition between the transition device and the outer surface of the portion of the UVC light source located below the top thereof, thereby minimizing any hinderances that may snag, obstruct or deflect the one or more UVC probes on any plant parts, trellis nets, poles or other obstructions in a growing area.

5. The one or more UVC probes of claim 1 further comprise a penetration device attached to the lower tip of the UVC light sources, wherein the penetration device comprises a curved or cone shaped lower tip to aid the UVC probe in penetrating into plant foliage in a substantially vertical orientation without causing damage to the plants, and thereby minimizing any hinderances that may snag, obstruct or deflect the one or more UVC probes on any plant parts, trellis nets, poles or other obstructions in a growing area.

6. The one or more UVC probes of claim 1 further comprise a penetration device attached to the lower tip of the UVC light sources, wherein the penetration device comprises a curved or cone shaped lower tip to aid the UVC probe in penetrating into plant foliage in a substantially vertical orientation without causing damage to the plants, and further comprises an upper transition surface on the top portion of the penetration device to create a relatively smoot and streamlined transition between the upper portion of the penetration device and the UVC light source.

7. The suspension device of claim 1 is an elongated hollow tube.

8. The means of conveying power to the one or more UVC probes of claim 1 is a power cord that attaches to the UVC light source, wherein the power cord is the suspension device.

9. The means of conveying power to the one or more UVC probes of claim 1 is a power cord that attaches to the UVC light source, wherein the power cord is the suspension device and further comprises a transition device attached around the top of the UVC light source and at least a portion of the power cord.

10. The plant disinfection apparatus of claim 1, wherein the elongated support device comprises an elongated beam configured to support two or more UVC probes, wherein the elongated beam is configured to be raised and lowered such that the one or more UVC probes are raised or lowered into the foliage of the one or more plants.

11. The plant disinfection apparatus of claim 1, wherein the elongated support device comprises an elongated beam configured to support two or more UVC probes, wherein the elongated beam is configured to be raised and lowered by one or more cables that are attached to a control unit, wherein the control unit is a motorized apparatus configured to move along a track system configured to be disposed above the one or more plants, such that the elongated beam can be raised and lowered along the path of the track system.

12. The plant disinfection apparatus of claim 1, wherein the elongated support device comprises an elongated beam configured to support two or more UVC probes, wherein the elongated beam is configured to be raised and lowered by one or more cables that are attached to a control unit, wherein the control unit comprises:
a motorized apparatus configured to move along a track system;
a mounting system configured to securely suspend, and slidingly engage the control unit with the track system;
a computer controllable motor attached to drive wheels, wherein the drive wheels engage the track system to allow the control unit to move along the track system;
a computer controllable motor attached to the one or more cable windings systems wherein the one or more cable winding systems are configured to engage the one or more cables wherein the elongated beam can be raised or lowered therein.

13. The plant disinfection apparatus of claim 1, wherein the elongated support device comprises an elongated beam configured to support two or more UVC probes, wherein the elongated beam is configured to be raised and lowered by one or more cables that are attached to a control unit, wherein the control unit comprises:
a motorized apparatus configured to move along a track system;
a mounting system configured to securely suspend, and slidingly engage the control unit with the track system;
a computer controllable motor attached to drive wheels, wherein the drive wheels engage the track system to allow the control unit to move along the track system;
a computer controllable motor attached to the one or more cable windings systems wherein the one or more cable winding systems are configured to engage the one or more cables wherein the elongated beam can be raised or lowered therein;
a retractable power cord system comprising a power cable configured to be attached to the control unit, and further comprising a tensioned reel apparatus comprising a motorized or spring tensioned reel configured to the attach to, and pull the power cable into the tensioned reel apparatus wherein the power cable is wound around the reel and tension is imparted on the power cable such that the power cable maintains tension between the retractable power cord system and the control unit as the control unit moves away and towards the retractable power cord system.

14. The plant disinfection apparatus of claim 1, wherein the elongated support device comprises an elongated beam configured to support two or more UVC probes, wherein the elongated beam is configured to be raised and lowered by one or more cables that are attached to a control unit, wherein the control unit comprises:
a motorized apparatus configured to move along a track system;
a mounting system configured to securely suspend, and slidingly engage the control unit with the track system;
a computer controllable motor attached to drive wheels, wherein the drive wheels engage the track system to allow the control unit to move along the track system;
a computer controllable motor attached to the one or more cable winding systems wherein the one or more cable winding systems are configured to engage the one or more cables wherein the elongated beam can be raised or lowered therein;
one or more anti-sway devices disposed adjacent to the control unit and also slidingly engaged with the track system, wherein the one or more anti-sway devices comprise vertical tracks that extend from the track system to the elongated beam, and wherein the elongated beam further comprises wheels or sliders that slidingly or rollingly engage with the vertical tracks such that the up and down movement of the elongated beam is stabilized by the vertical tracks.

15. A power feed system to supply electrical power to a moving apparatus, the power feed system comprising:
a retractable power cable apparatus comprising an output power cable configured to be attached to a moving apparatus, the retractable power cable apparatus comprising a tensioned reel apparatus with a motorized or spring tensioned reel configured to the attach to, and pull the output power cable into the tensioned reel apparatus and wind the output power cable around a reel, wherein tension is imparted on the output power cable;
wherein the output power cable maintains tension between the retractable power cable apparatus and the moving apparatus as the moving apparatus moves away and towards the retractable power cord system.

16. The retractable power cable apparatus of claim 15 is disposed in a fixed location and further comprises one side with a port that allows the output power cable to enter the retractable power cable apparatus located on that side, and wherein the moving apparatus is configured to move in a direction away and towards that side of the retractable power cable apparatus wherein the output power cable retains a substantially direct pathway between the retractable power cable apparatus and the attachment point on the moving apparatus.

17. The retractable power cable apparatus of claim 15 is configured to pivot around at least one axis and comprises a port that allows the output power cable to enter the retractable power cable apparatus, wherein the output power cable is configured to move in a direction along the pivot axis, and wherein the output power cable retains a substantially direct pathway between the retractable power cable apparatus and the attachment point on the moving apparatus.
